(19) United States
(12) Patent Application Publication
(10) Pub. No.: US 2007/0049637 A1
(43) Pub. Date: Mar. 1, 2007

(54) Process for the synthesis of cationic lipids

(75) Inventors: Lorenzo De Ferra, Patrica (IT); Mauro Anibaldi, Patricia (IT); Ettore Ammirati, Patricia (IT)

Correspondence Address: NIXON & VANDERHYE, PC, 901 NORTH GLEBE ROAD, 11TH FLOOR, ARLINGTON, VA, 22203, US

(73) Assignee: CHEMI SPA, Cinisello Balsamo (IT)

(21) Appl. No.: 10/579,223

(22) PCT Filed: Nov. 3, 2004
(86) PCT No.: PCT/EP04/52788

§ 371 Date: May 12, 2006

(30) Foreign Application Priority Data
Nov. 12, 2003 (IT) MI2003 A 002185

(51) Int. Cl. A61K 31/22; C07C 229/02

(52) U.S. Cl. 514/547; 554/107

(57) ABSTRACT

A process for the synthesis of lipid cations having general formula (6): 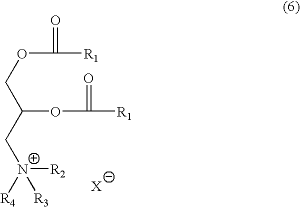
in which: R₁ represents a lipophilic chain; R₂, R₃, R₄, which are identical or different from one another, represent C₁-C₁₀ alkyl, C₁-C₁₀ alkenyl, or C₁-C₁₀ alkynyl radicals, optionally containing hydroxyl, ether, halogen and acyloxy functions, and X₋ is an oxy-anion or a halide; in which a compound of formula (2), 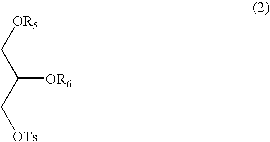
in which R₅ and R₆, which are identical or different from one another, represent a C₁-C₅ acyl, a benzyl group or a diol-protective group, is reacted in an alcoholic solvent with from 1 to 6 equivalents of NR₂R₃R₄.

The subject of the present invention is a synthesis, which is economical, safe and can be implemented on a large scale, of lipid cations having the general formula given in FIG. 1: 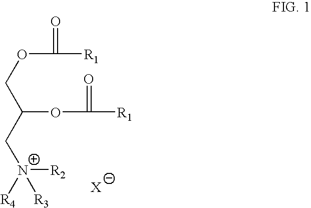
in which R₁ represents a lipophilic chain, preferably selected from C₁-C₂₄ alkyl, C₁-C₂₄ alkenyl, C₁-C₂₄ alkynyl, C₁-C₂₄ alkanoyl, and C₁-C₂₄ alkenoyl or alkynoyl radicals;
R₂, R₃, R₄, which are identical or different from one another, represent C₁-C₁₀ alkyl, C₁-C₁₀ alkenyl, or C₁-C₁₀ alkynyl radicals, optionally containing hydroxyl, ether, halogen and acyloxy functions, and
X⁻ is an oxy-anion or a halide.

In particular, the subject of the invention is the synthesis of N-[1-(2, 3-dioleyloxy)propyl]-N,N,N-trimethylammonium chloride (DOTAP-Cl) the structure of which is given in FIG. 2:

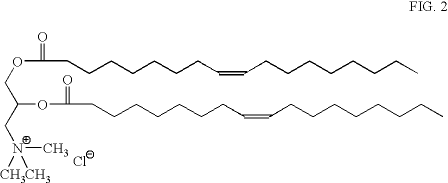

DOTAP-Cl is a compound which belongs to the cytofectin class. These amphiphilic, cationic molecules are usable in the gene therapy field and, in particular, in the field of transfection (the process by means of which exogenous DNA or RNA fragments are transported through the cell membranes of host cells).

Various strategies have been adopted for achieving this objective, some of which are based on the use of recombinant viruses as vectors. However, the above-mentioned techniques may encounter various problems such as, for example, problems of an immunological nature which are due to the presence of antibodies in the host organisms, or problems of an epidemiological nature which are correlated with the possibility, albeit remote, of thus creating a new mutant virus that is capable of becoming potentially infective and therefore dangerous.

To try to avoid these risks, alternative techniques have been developed, and are based precisely on the formulation of liposomal complexes based on lipid cations containing nucleic acids or, more generally, pharmacologically active molecules which can “fuse” with the cell membranes, releasing their content into the cytoplasm (hence the name cytofectin).

Even though the mechanism of their operation on a molecular basis still remains unknown in detail, these complexes have been found effective in the transportation and in the intracellular release of gene material and active ingredients.

### FIELD OF THE INVENTION

DOTAP, that is, N-[1(2,3-dioleyloxy)propyl]-N,N,N-trimethylammonium, is one of the cationic lipids which is most often used in liposome formulations containing nucleic acids; for this reason, various approaches to the synthesis of this molecule are reported in the literature; for example, in U.S. Pat. No. 5,925,623 (M. H. Nantz; M. J. Bennet et al,), the synthesis method given below in Scheme 1 is described:

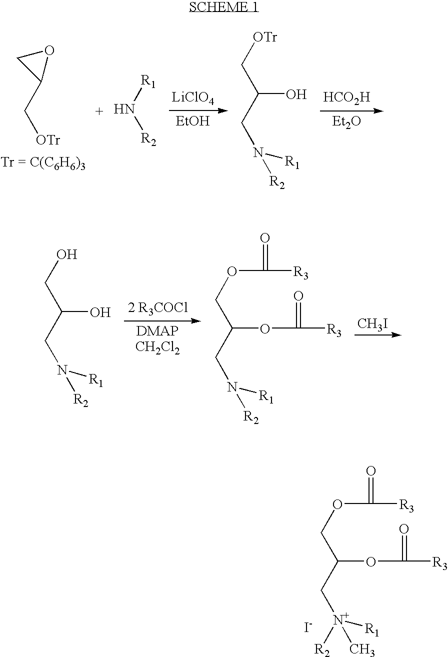

This synthesis represents a very flexible approach for the preparation of a whole family of cationic lipids; to bring about the quaternization of the nitrogen atom, however, this method provides for the use of methylating agents which are very toxic and potentially carcinogenic (such as, for example, iodomethane) and hence difficult to use in large-scale preparations.

In Example 5 of U.S. Pat. No. 5,264,618 (P. L. Feliner; R. Kumar, C. Basava et al.), the synthesis method given below in Scheme 2 is described:

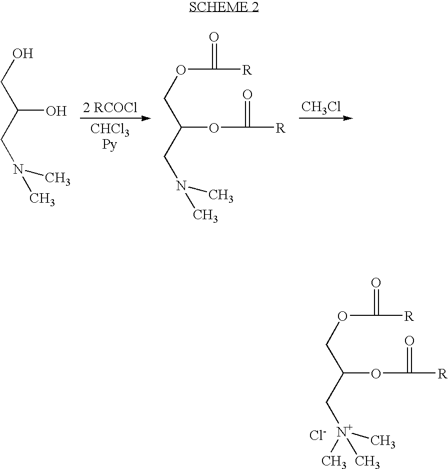

However toxic methylating reagents (in this case chloromethane) are also used in this synthesis, so that problems similar to those of the previous preparation are encountered when the method is implemented on a large scale.

Other syntheses of DOTAP described in the literature are those proposed by L. Stratamatos, et al., Biochemistry 27:3917-3925 (1988) given in Scheme 3: 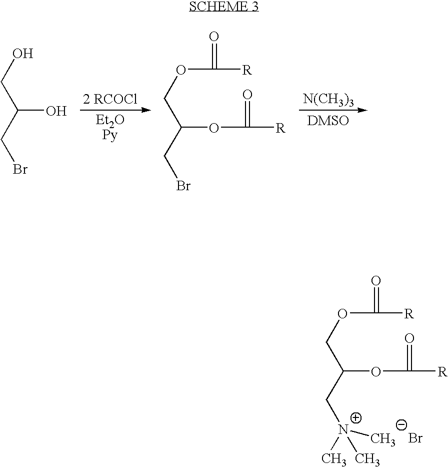
and that which provides for the same intermediates but uses different reagents, and which is described in German patent application DE4013632A1 (H. Weickmann; K. Fincke et al.), given in scheme 4:

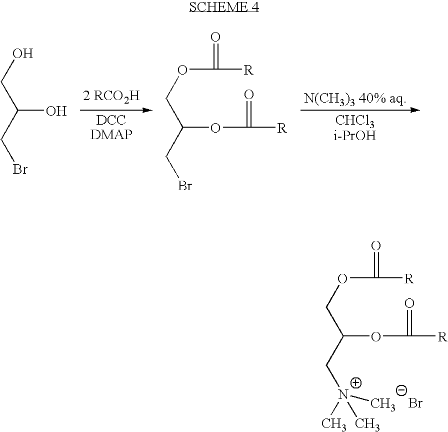

Although these syntheses do not provide for the use of toxic and dangerous reagents, they are not advantageous for the implementation of industrial preparations since they start with a raw material (bromopropandiol) which is quite expensive; the purification of the final product is also performed by means of a complex and expensive technique such as chromatography on silica gel which, moreover, is also provided for in the syntheses indicated above in Schemes 1 and 2.

In the light of the documents of the literature, it is therefore clear that there is no process for the synthesis of DOTAP which can be performed easily on a large scale.

### DESCRIPTION OF THE INVENTION

The subject of the present invention is a process for the large-scale synthesis of DOTAP-Cl (and more generally of cationic lipids of similar structure given in FIG. 1) from raw materials which are easily available and inexpensive; this process can be summarized by Scheme 5 given below, in which R₁, R₂, R₃, R4 and X⁻ have the meanings listed above and in which R₅ and R₆, which are identical or different from one another, represent a C₁-C₅ acyl, a benzyl group or a diol-protective group.

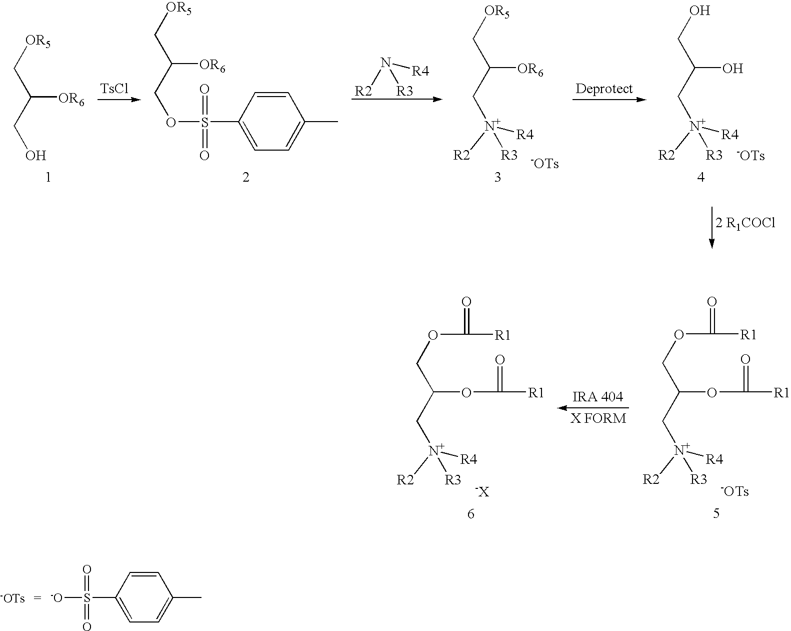

One of the most innovative aspects of the process of the invention is the successful synthesis, in a simple manner and without the aid of toxic and/or excessively expensive reagents, of the intermediate having the general formula indicated in FIG. 3: 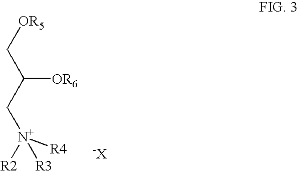
in which R₂, R₃, R₄R₅, R₆ and X⁻ have the meanings given above.

The term “diol-protective group” means one of the protective groups normally used for the protection of 1,2- and 1,3-diols; these protective groups are well known in the art and are described, for example, in Greene et al. Protective Groups in Organic Synthesis, Third Edition, John Wiley & Sons, 1999, which is incorporated herein by reference. Amongst the various protective groups described in the above-mentioned book, those which are preferred for the implementation of the present invention are the cyclic ketals; in greater detail, according to the preferred embodiment of the invention, R₅ and R₆ together represent isopropylidene; in that case, when R₂, R₃, R₄ are methyl radicals, the compound adopts the preferred structural formula given in FIG. 3bis. 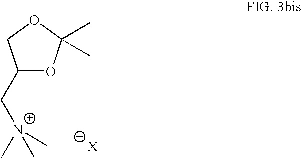
in which X⁻ has the meaning given above.

Various methods are reported in the literature for the preparation of compounds which can be represented by FIG. 3 such as, for example, those described in U.S. Pat. No. 6,084,131 (R. I. Hollingsworth; G Wang) or in D. J. Triggle, B. Belleau Can. J. Chem. 40: 1201-1215 (1962); however, these syntheses provide for the quaternization of the amine function by means of highly toxic and potentially carcinogenic methylating agents and are therefore difficult to implement on an industrial scale. Alternative synthesis methods which do not require the use of methylating agents are described by S. L. Morris-Natschke; K. L. Meyer Journal Med. Chem. 33 (6): 1812-1818 (1990 and by D. A. Jaeger; J. Mohebalian; P. L. Rose Langmuir 6: 547-554 (1990); however, these synthesis methods are of little industrial interest since they are very laborious and are characterized by very low yields.

The process of the present invention, on the other hand, enables the intermediate of FIG. 3 to be prepared on a large scale and with high yields in a surprisingly simple and economic manner; the product thus obtained is also sufficiently pure to be usable without further purification.

### DETAILED DESCRIPTION OF THE INVENTION

The first step of the process according to the present invention consists in the preparation of the tosylate derivative of formula (2); this reaction is preferably carried out by reacting from 0.9 to 1.2 equivalents of compound of formula (1) with 1 equivalent of tosyl halide in an apolar, organic solvent, preferably a hydrocarbon, even more preferably toluene; this reaction is normally carried out at a temperature of 15-35° C., even more preferably at 20-25° C., with the use of from 0.8 to 1.2 litres of solvent per equivalent of substrate 1. The compound (2) thus obtained is brought to residue in accordance with conventional techniques, preferably by distillation at reduced pressure.

In the second step, 1 equivalent of intermediate (2) is reacted with from 1 to 6 equivalents of NR₂R₃R₄, in which R₂, R₃ and R₄, which are identical or different from one another, have the meanings given above and, preferably, all three are methyl radicals. The reaction is carried out in an alcoholic solvent, preferably ethanol, isopropanol, or methanol, operating at a temperature of 50-100° C.; the solvent is preferably used in quantities of from 0.5-1.5 litres per equivalent of NR₂R₃R₄. The compound (3) thus obtained is brought to residue in accordance with conventional techniques, preferably by distillation at reduced pressure.

In the third step, the diol-protective group is removed to give intermediate (4) with the use of the techniques that are known from the literature; if the protective group is a ketal, the removal will preferably take place by acid hydrolysis.

To obtain compound (5), compound (4) is suspended in an aprotic, apolar, organic solvent, preferably in a chlorinated solvent such as, for example, methylene chloride, chloroform, or tetrachloroethylene; the solvent is preferably used in quantities of 3.5-5.5 litres per equivalent of compound (4). From 2 to 4 equivalents of R₁COCl, where R₁ has the meaning given above, are then added and reacted at a temperature of 35-45° C. The compound (5) thus obtained is brought to residue in accordance with conventional techniques, preferably by distillation at reduced pressure. The last step is represented by ion exchange of the tosylate anion of compound (5) with the halide anion of compound (6); this exchange is preferably performed by chromatography on strong basic ion-exchange resin. Finally, the final purification of the product is performed by a simple sequence of crystallizations, rendering the process easier to implement on an industrial scale.

According to the preferred embodiment of the invention, solketal tosylate (2) is obtained from isopropylidene glycerol (about 1.1 equivalents) by condensation with tosyl chloride (about 1.0 eq.) in toluene at about 25° C. for 2 hours in the presence of triethylamine (about 1.0 equivalent) used as a scavenger for the hydrochloric acid which is formed as a reaction by-product. Upon completion of the reaction and when 3 washings with water have been performed to remove the triethylammonium chloride and the excess solketal, the organic phase is brought to residue to give product (2) in a practically quantitative manner and ready for use in the subsequent reaction without further purification.

The solketal tosylate (2) (about 1.0 equivalent), 40% aqueous trimethylamine (from 3 to 4 equivalents, preferably about 4 equivalents), and an equal volume of methanol are then loaded into an autoclave and left with stirring at a temperature of from 65-85° C., preferably about 75° C., for a period of from 8 to 24 hours, preferably about 16 hours. Upon completion of the reaction, the excess trimethylamine (which is captured by salifying it in a 10% hydrochloric acid trap) and the solvents are removed at reduced pressure to give a pale yellow, creamy solid which is taken up with water and decolorized with adsorbent carbon (10% p/p). After stirring at ambient temperature for about 1 hour, the active carbon is filtered out and the aqueous solution containing the 2,2-dimethyl-4-trimethylammonium methyl-1,3-dioxolane tosylate (3) is ready for the next synthesis step.

The hydrolysis of the acetonide which is present as a protective group in compound 3 is performed in water by acidification, preferably with p-toluene sulphonic acid, to a pH of from 1 to 3, preferably pH=1.5, at a temperature of from 30° C. to 50° C., preferably at 40° C., for a period of from 2 to 5 hours, preferably 3.5 hours. Upon completion of the reaction, the pH is returned to neutrality with 6M NaOH, the solution is concentrated to residue at reduced pressure, and the residue is taken up with MIBK (5 volume/weight) and is dehydrated by azeotropic distillation with Dean-Stark apparatus. Upon completion of the removal of the water, the 1-2,dihydroxy-trimethylammonium propane tosylate (4) is obtained as a pale yellow, waxy solid, simply by concentration at reduced pressure, ready for the next synthesis step.

To obtain DOTAP tosylate (5), compound (4) is suspended in methylene chloride with the use of from 8 to 15 volumes/weight, preferably 13 volumes/weight, and from 2 to 4 equivalents, preferably 3 equivalents, of dimethylaminopyridine (DMAP) are added; oleoyl chloride is then added (in equal equivalents relative to the DMAP), whilst the temperature is kept at about 30° C.; upon completion of the addition, the mixture is heated to 35-45° C., preferably to 40° C., for a period of from 2 to 5 hours, preferably 3.5 hours. The reaction is then stopped with 17 volumes of methanol and stirring is continued for about 30 minutes; 2 extractions are then performed with water at about pH=3, and the last with brine at about pH=6; the organic phase is brought to residue at reduced pressure, thus giving the crude DOTAP tosylate (5).

The next step to obtain the DOTAP-Cl is represented by the ion exchange of the counter ion, which is performed by eluting the DOTAP-OTs dissolved in methanol (about 3 volumes/weight) in a chromatography column containing a strong basic ion-exchange resin in chloride form (from 2 to 8 equivalents of resin per mole of product); in the most preferred embodiment, 5 equivalents/mole of Amberlite IRA 404 resin, Cl form (Rohm & Haas) are used.

Finally, the final purification of the product is performed in accordance with conventional techniques and, in particular, by crystallization, preferably from acetonitrile.

The following examples serve to explain the implementation of the present invention in even greater detail and are purely illustrative and not limiting thereof.

### EXAMPLES

### Example 1

### (Synthesis of (R,S) solketal tosylate-2)

8.85 g (73 mmol) of dimethylaminopyridine (DMAP), 74 g (733 mmol) of triethylamine (TEA), 100 g (757 mmol) of (R,S) solketal, and 420 ml of toluene were loaded into a 4-necked, 2-litre flask, provided with a mechanical stirrer and a reflux condenser, under a moderate stream of nitrogen. Stirring was started at T=20/25° C. When a homogeneous solution had been obtained, a solution prepared previously by dissolving 142 g (745 mmol) of p-toluene sulphonyl chloride in 340 ml of toluene in an Erlenmeyer flask was added dropwise from a dropping funnel over a period of approximately 1 h. Stirring was continued at the same temperature for 2 hours and the reaction was then stopped by adding 715 ml of water over a period of 10 minutes and continuing to stir for a further 15 minutes. After the reaction mixture had been transferred to a separating funnel, the phases were separated. 650 ml of water and 14 ml of 33% HCl were added to the organic phase, the mixture was agitated and left to clear, and the phases were separated. 700 ml of water was added to the organic phase, the mixture was agitated, and the phases were separated. A further extraction was then performed with a solution of 70 g of sodium bicarbonate dissolved in 650 ml of water and, finally, the organic phase was extracted for a last time with 700 ml of water. The organic phase thus obtained was concentrated at reduced pressure to give 195 g (682 mmol) of compound 2 in the form of a yellowish oil (yield 90%).

In TLC (silica gel), product 2 migrated with an Rf=0.9 when eluted with CHCl₃/Acetone=85/15 and developed with ammonium molybdate cerium sulphate.

### Example 2

### (Synthesis of (R,S) 2,2-dimethyl-4-trimethylammonium methyl-1,3-dioxolane tosylate-3)

91 g (0.318 mol) of (R,S) solketal tosylate (2), 205 ml of methanol, and 205 ml of 40% aqueous trimethylamine were loaded into a 600 ml autoclave, the autoclave was closed, mechanical stirring was started, and the mixture was heated to T=75° C.; when this temperature had been reached, an internal pressure of 1.2 bar was measured by means of a manometer. Stirring was continued, the temperature was maintained for 16 hours and the contents of the autoclave were then transferred into a 1-litre, 4-necked flask provided with a mechanical stirrer and a Liebig condenser with a 500 ml collecting flask. Distillation was started at reduced pressure, during which the vapours coming from the collecting flask were passed through a 10% hydrochloric acid trap to destroy the excess trimethylamine.

Upon completion of the distillation, the residue obtained was dissolved in 300 ml of distilled water and 9 g of decolorizing carbon was added with stirring, stirring was continued for 1 hour at ambient temperature and, finally, the carbon was filtered out on a Dicalite panel. The solution thus obtained was ready for subsequent hydrolysis to give product 3; removal of the water at reduced pressure sufficed to give 105 g (0.304 mol) of a pale yellow, waxy solid (yield 96%).

In TLC (silica gel), product 3 migrated with an Rf=0.4 when eluted with CH₃OH/H₂O/CH₃COOH=10/0.25/0.25 and developed with ammonium molybdate cerium sulphate.

¹H NMR (200 MHz, D₂O)) δ 7.7-7.3 (dd, 4H, Ph); 4.65 (m,H, OCHCH₂); 4.2 (dd, 1H, H′CHN(CH₃)₃); 3.65 (dd, 1H, HCH″N(CH₃)₃); 3.43 (m, 2H, OCH₂CH); 3.1 (s, 9H, N(CH₃)₃) 2.36 (s, 3H, Ph-CH₃); 1.45-1.40 (2s, 6H, CH₃CCH₃).

### Example 3

### (Synthesis of (R,S)1,2-dihydroxy-trimethylammonium propane tosylate-4)

105 g (0.304 mol) of (R,S)2,2-dimethyl-4-trimethylammonium methyl-1,3-dioxolane tosylate (3) was dissolved in 600 ml of distilled water, the solution was loaded into a 3-necked flask provided with a magnetic stirrer and an immersed pH meter, stirring was started, and a 10% p-toluene sulphonic acid solution was added to reach pH=1.5; upon completion of the addition, the mixture was heated to T=40° C., and that temperature was maintained for 3.5 hours. When that time had elapsed, the solution was returned to ambient temperature and a 10% NaOH solution was added until pH=6 was reached. The solution obtained was brought to residue at reduced pressure; finally, the residue was dehydrated by taking it up with 800 ml of methyl isobutyl ketone (MIBK) and performing an azeotropic distillation with a Dean-Stark apparatus. 92 g (0.301 mol) of 1,2-dihydroxy-trimethyl-ammonium propane tosylate was obtained as a yellowish, waxy solid, with a practically quantitative yield.

¹H NMR (200 MHz, D₂O) δ 7.7-7.3 (dd, 4 H, Ph); 4.2 (m, 1H, OCHCH₂); 3.55 (m, 2H, CH₂N(CH₃)₃); 3.40 (m, 2H, OCH₂CH); 3.1 (s, 9H, N(CH₃)₃) 2.36 (s, 3H, Ph-CH₃).

### Example 4

### (Synthesis of (R,S)1,2-dioleoyl-trimethylammonium propane tosylate-5)

92 g (0.301 mol) of 1,2-dihydroxy-trimethylammonium propane tosylate (4), 105 g (0.85 mol) of dimethylaminopyridine (DMAP), and 1.35 litres of CH₂Cl₂ were loaded into a 2-litre, 4-necked flask provided with a mechanical stirrer and a reflux condenser, and with a moderate stream of nitrogen, and stirring was performed until a homogeneous solution was obtained; 246 g (0.82 mol) of oleyl chloride was added dropwise thereto over a period of about 20 minutes, whilst the temperature was controlled with a cold bath so as not to exceed 30° C. Upon completion of the addition, stirring was continued for 3.5 hours. Upon completion of the reaction, 1.75 litres of CH₃OH was added at ambient temperature and stirring was continued for 30 minutes; 1.75 litres of distilled water was then added and 33% HCl (25g) was added with stirring to reach pH3/3.5 of the aqueous phase; stirring was stopped and the whole was transferred to a 5-litre separating funnel. The phases were separated. 870 ml of distilled water and 870 ml of CH₃OH were added to the lower, organic phase, vigorous agitation was performed, the mixture was left to clear, and the phases were separated. 870 ml of distilled water and 870 ml of CH₃OH were added to the lower, organic phase and 10% NaOH (1 g) was added with stirring to reach pH 6 of the aqueous phase; 30 ml of a saturated NaCl solution was added, still with stirring, and the mixture was then left to clear and the phases were separated. The rich, lower, organic phase was concentrated to residue giving 264 g; the HPLC titre of the DOTAP-OTs (5) in this crude product, against an external standard, was 66% (174 g 0.21 mol) yield 69%.

In TLC (silica gel), product 5 migrated with an Rf=0.4 when eluted with CHCL₃/acetone/CH₃OH/CH₃COOH/H₂O=50/15/5/5/2 and developed with copper sulphate reagent.

### Example 5

### (Ion exchange of the anion from DOTAP tosylate to DOTAP chloride-6)

50 g of crude DOTAP-OTs (obtained in Example 4-titre 66%, 39.6 mmol) was dissolved in 150 ml of methanol; this solution was loaded into a chromatography column packed with 230 ml of strong basic IRA 404 ion-exchange resin, chloride form (produced by Rohm & Haas), previously washed with 1.2 l of distilled water and placed in a methanol environment (1.2 l). The methanolic solution containing the DOTAP-OTs was then eluted by gravity with a flow of 2.5 m./minute, a dead volume of about 50 ml being discarded. Finally, the elution of the product with methanol was completed, and a single fraction of about 600 ml was collected. This solution was concentrated in a Rotavapor apparatus to a volume of 100 ml and then 100 ml of acetonitrile was added to this solution and brought to an oily residue (44.0 g) in the Rotavapor apparatus. This residue was crystallized from acetonitrile at 20° C. 20.7 g (29.7 mmol) of DOTAP-Cl, yield 75% was obtained after drying overnight under high vacuum at ambient T.

In TLC (silica gel), product 6 migrated with an Rf=0.4 when eluted with CHCL₃/acetone/CH₃OH/CH₃COOH/H₂O=50/15/5/5/2 and developed with copper sulphate reagent.

¹H NMR (200 MHz, CDCl₃) δ5.6 (m Broad, 1H, OCHCH₂); 5.28 (m, 4H, 2× CH═CH); 4.5 (t, 2H, OCH₂CHO); 4.05 (dd, 1H, H′CHN(CH₃)₃); 3.7 (dd, 1H, HCH″N(CH₃)₃); 3.47 (s, 9H, N(CH₃)₃) 2.26 (m, 4H, 2× CH₂COO); 1.9 (m, 8H, 4× CH₂CH═CH); 1.5 (m, 4H, CH₂CH₂COO); 1.4-1.2 (m, 36H, CH₂ aliphatic); 0.82 (dt, 6H, CH₂CH₃).

¹³C NMR (200 MHz, CDCl₃) δ 173.1, 172.7, 130.0, 129.9, 129.6, 129.5, 65.9, 65.7, 63.2, (3) 54.2, 34.1, 33.9, 31.9, 29.7, 29.5, 29.3, 29.2, 29.17, 29.14, 29.09, 27.2, 27.1, 24.7, 24.6, 22.6, 14.1.

1-21. (canceled)

22. A process for the synthesis of lipid cations having general formula (6): 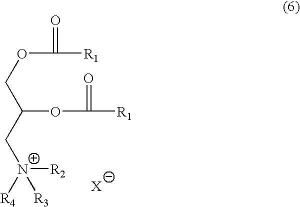
in which: R₁ represents a lipophilic chain, preferably selected from C₁-C₂₄ alkyl, C₁-C₂₄ alkenyl, C₁-C₂₄ alkynyl, C₁-C₂₄ alkanoyl, and C₁-C₂₄ alkenoyl or alkynoyl radicals, R₂, R₃, R₄, which are identical or different from one another, represent C₁-C₁₀ alkyl, C₁-C₁₀ alkenyl, or C₁-C₁₀ alkynyl radicals, optionally containing hydroxyl, ether, halogen and acyloxy functions, and X⁻ is an oxy-anion or a halide, characterized in that a compound of formula (2), 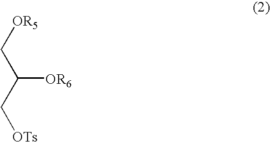
in which R₅ and R₆, which are identical or different from one another, represent a C₁-C₅ acyl, a benzyl group or a diol-protective group, is reacted in an alcoholic solvent with from 1 to 6 equivalents of NR₂R₃R₄, in which R₂, R₃ and R₄ have the meanings given above, to give the compound of formula (3) 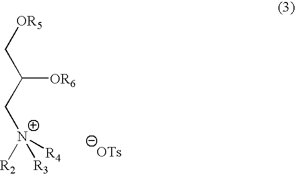
in which R₂, R₃, R₄, R₅ and R₆ have the meanings given above.

23. A process according to claim 22, characterized in that the alcoholic solvent is selected from ethanol, isopropanol and/or methanol.

24. A process according to claim 22, characterized in that the alcoholic solvent is used in a quantity of from 0.5-1.5 litres per equivalent of NR₂R₃R₄.

25. A process according to claim 22, characterized in that all three of R₂, R₃ and R₄ are methyl radicals.

26. A process according to claim 22, characterized in that it is carried out at a temperature of from 50-100° C.

27. A process according to claim 22, characterized in that the diol-protective group is a ketal, preferably a cyclic ketal, even more preferably a solketal.

28. A process according to claim 22, characterized in that the compound of formula (2) is obtained by reacting from 0.9 to 1.2 equivalents of compound of formula (1). 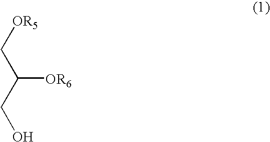
with 1 equivalent of tosyl halide, preferably chloride, in an apolar, organic solvent.

29. A process according to claim 28, characterized in that the apolar, organic solvent is a hydrocarbon, preferably toluene.

30. A process according to claim 28, characterized in that the reaction is carried out at a temperature of 15-35° C., preferably 20-25° C.

31. A process according to claim 28, characterized in that the reaction is carried out with the use of from 0.8 to 1.2 litres of solvent per equivalent of compound (1).

32. A process according to claim 22, comprising the removal of the R₅ and R₆ groups to give compound (4) 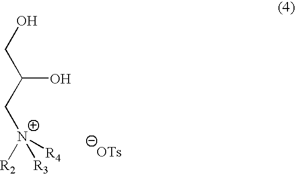
in which R₂, R₃ and R₄ have the meanings given above, the subsequent reaction of compound (4) with 2-4 equivalents of R₁COCl in an aprotic, apolar, organic solvent, preferably a chlorinated solvent, in which R₁ has the meaning given above, to give compound (5) 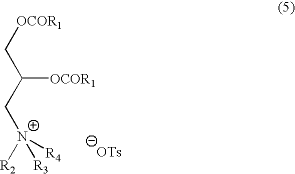
in which R₁, R₂, R₃ and R₄ have the meanings given above, and the subsequent ion exchange of the tosylate anion of compound (5) with a halide anion to give the lipid cation of formula (6).

33. A process according to claim 32, characterized in that groups R₅ and R₆ are removed by acid hydrolysis.

34. A process according to claim 32, characterized in that the aprotic, apolar, organic solvent is used in a quantity of 3.5-5.5 litres per equivalent of compound (4).

35. A process according to claim 32, characterized in that the aprotic, apolar, organic solvent is selected from methylene chloride, chloroform, and tetrachloroethylene.

36. A process according to claim 32, characterized in that the ion exchange is performed by chromatography on ion-exchange resin.

37. A process according to claim 36, characterized in that the ion-exchange resin is a strong basic resin.

38. A process according to claim 22, characterized in that the lipid cation of formula (6) is purified by crystallization, preferably from acetonitrile.

39. A process according to claim 22, characterized in that the lipid cation of formula (6) is N-[1-(2,3-dioleyloxy-propyl]-N,N,N-trimethylammonium chloride (DOTAP-Cl).

40. A compound of formula (4) 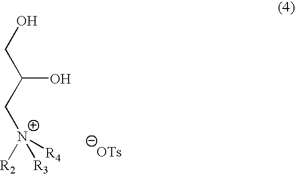
in which R₂, R₃ and R₄, which are identical or different from one another, represent C₁-C₁₀ alkyl radicals, C₁-C₁₀ alkenyl radicals, or C₁-C₁₀ alkynyl radicals, optionally containing hydroxyl, ether, halogen, and acyloxy functions.

41. A compound of formula (5) 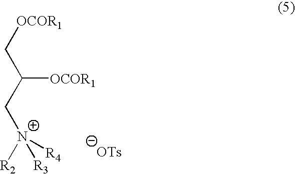
in which: R₁ represents a lipophilic chain, preferably selected from C₁-C₂₄ alkyl, C₁-C₂₄ alkenyl, C₁-C₂₄ alkynyl, C₁-C₂₄ alkanoyl, and C₁-C₂₄ alkenoyl or alkynoyl radicals, and R₂, R₃, R₄, which are identical or different from one another, represent C₁-C₁₀ alkyl, C₁-C₁₀ alkenyl, or C₁-C₁₀ alkynyl radicals, optionally containing hydroxy, ether, halogen and acyloxy functions.

42. Use of compounds of formula (4) as intermediates in the synthesis of cationic lipids having general formula (6): 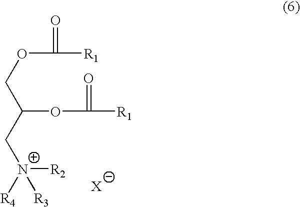
in which R₁ represents a lipophilic chain, preferably selected from C₁-C₂₄ alkyl, C₁-C₂₄ alkenyl, C₁-C₂₄ alkynyl, C₁-C₂₄ alkanoyl, and C₁-C₂₄ alkenoyl or alkynoyl radicals, R₂, R₃, R₄, which are identical or different from one another, represent C₁-C₁₀ alkyl, C₁-C₁₀ alkenyl, or C₁-C₁₀ alkynyl radicals, optionally containing hydroxyl, ether, halogen and acyloxy functions, and X⁻ is an oxy-anion or a halide.

43. Use of compounds of formula (5) as intermediates in the synthesis of cationic lipids having general formula (6): 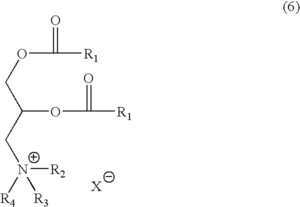
in which R₁ represents a lipophilic chain, preferably selected from C₁-C₂₄ alkyl, C₁-C₂₄ alkenyl, C₁-C₂₄ alkynyl, C₁-C₂₄ alkanoyl, and C₁-C₂₄ alkenoyl or alkynoyl radicals, R₂, R₃, R₄, which are identical or different from one another, represent C₁-C₁₀ alkyl, C₁-C₁₀ alkenyl, or C₁-C₁₀ alkynyl radicals, optionally containing hydroxyl, ether, halogen and acyloxy functions, and X⁻ is an oxy-anion or a halide.
